(19) United States
(12) Patent Application Publication
(10) Pub. No.: US 2026/0069219 A1
(43) Pub. Date: Mar. 12, 2026

(54) Modular Video Playback Facility for a Patient on a Patient Table in a Magnetic Resonance Facility, and Magnetic Resonance Facility

(71) Applicant: Siemens Healthineers AG, Forchheim (DE)

(72) Inventors: Johann Sukkau, Herzogenaurach (DE); Volker Matschl, Bamberg (DE); Hans Weber, Erlangen (DE); Vincent Kelber, Bamberg (DE)

(73) Assignee: Siemens Healthineers AG, Forchheim (DE)

(21) Appl. No.: 19/321,397

(22) Filed: Sep. 8, 2025

(30) Foreign Application Priority Data
Sep. 9, 2024 (EP) 24199110.8

(51) Int. Cl. A61B 5/00; G01R 33/28

(52) U.S. Cl. CPC A61B 5/7445; G01R 33/283

(57) ABSTRACT

The disclosure relates to a modular video playback system for use in a magnetic resonance device. The video playback system comprises a display module with a respective display unit for a left and right eye of the patient, each display unit including a display, a support arrangement for holding the display at a distance from the head laterally, and an optical arrangement fastened to the support arrangement at least partially in front of each respective eye for projecting an image output by the display onto the patient's eye. The video playback system also includes a holder that may be detachably connected to the display module and has fastening means for detachably fastening to at least one component of the patient table, a data transmission arrangement which can be connected to the display module, and a power supply arrangement which can be connected to the display module.

### CROSS-REFERENCE TO RELATED APPLICATIONS

The present application claims priority to and the benefit of European patent application no. EP 24199110.8, filed on Sep. 9, 2024, the contents of which are incorporated herein by reference in their entirety.

### TECHNICAL FIELD

The disclosure relates to a modular video playback facility for a patient on a patient table in a magnetic resonance facility, and to a magnetic resonance facility.

### BACKGROUND

Patient receptacles in magnetic resonance facilities for medical imaging are often relatively narrow. In addition, there is a high level of noise inside the patient receptacle during the imaging process, and magnetic resonance imaging procedures are usually lengthy, typically ranging from 10 minutes to half an hour. Therefore, it has already been proposed in the prior art to provide multimedia entertainment for a patient positioned on a patient table inside the receptacle of a magnetic resonance facility, in particular for an imaging procedure. For example, it is known to use headphones that simultaneously provide noise protection and deliver audio content.

With regard to delivering video content, various solutions have also been proposed in the prior art. For example, it is known to provide a mirror above the patient in their line of sight when in the supine position in order to redirect said line of sight from vertical to horizontal, thereby allowing the patient to view a magnetic-resonance-compatible monitor located outside the patient receptacle, for example on a rear side of a shielded enclosure for the main magnet unit. In particular, such a mirror, which can be angled at 45°, for example, can be mounted on a head coil arranged around the head of the patient. Such an arrangement is described, for example, in DE 10 2013 217 561 B4. However, the problem here is that the distance between the image source, i.e. the monitor, and the eyes of the patient depends on the table position. This means that the size of the image also varies considerably depending on the table position and therefore on the body part currently being examined. This is disadvantageous in terms of image perception and accommodation by the eyes.

Another system known as “Innovision” has also been proposed, in which the mirror, which is again angled at 45°, has a parabolic shape in order to magnify a video image behind the head of the patient, which image is generated by a video projector integrated into an Innovision device. This requires using a heavy device inside the patient receptacle. Due to the height of the device, the vertical travel of the patient table must be mechanically limited so that the device is not damaged by accidental excessive upward movement of the patient table and a resulting collision of the device with the wall of the patent receptacle of the main magnet unit. Therefore, a video system with significantly reduced height would be desirable.

It has also been proposed to provide the patient with a video headset, as proposed, for example, in DE 10 2007 030 972 B3. However, a video headset is awkward to use, as it must first be put on by the patient, which increases the preparation time for a magnetic resonance examination. Moreover, the video headset isolates the patient from the external environment, which is not acceptable to some patients. In addition, a cable for power supply and signal transmission is required which must be connected during the preparation time. Problems may also arise with regard to magnetic resonance compatibility. A major concern is sterility/hygiene, as direct contact with the skin of the patient necessitates cleaning after each patient. This involves additional time and effort, and components such as a rubber rim on the video headset can be damaged, which increases wear and tear. A device without direct skin contact is therefore desirable.

Other approaches involving some complexity in terms of implementation and resources required have also been proposed, such as the use of a bundle of coherent optical fibers, as described in U.S. Pat. No. 4,901,141 A, and the use of relay lenses with liquid crystal displays, see for example U.S. Pat. No. 5,877,732 A.

Another problem with the known approaches is that they often cannot be used in all conceivable positions of the patient. For example, approaches that use a mirror to deflect the line of sight to a monitor located in particular behind the patient can only be used in the supine position. If the mirror is mounted on the head coil, it can only be used in the position in which the patient is moved head-first into the patent receptacle (“head-first supine”—HFS). By contrast, the solution involving a video headset can also be used in a prone position, for example for a mammography examination, or in a side-lying (lateral) position, for example for a prostate examination.

### SUMMARY

It would therefore be desirable to have a magnetic-resonance-compatible, lightweight, and self-contained video system that can be used for as many patient positionings as possible, which does not completely isolate the patient from the external environment, that can be positioned quickly in the field of view, and that avoids direct skin contact. Magnetic resonance compatibility is to be understood as meaning that the video playback facility should be, as far as possible, non-magnetic and should generate no, or as few as possible, magnetic resonance image artifacts. Self-contained means that both the power supply and data transmission should preferably not require cabling. Not isolating the patient from the external environment is to be understood as meaning that, at least optionally, the patient should be able to at least partially perceive their surroundings. Usability in all patient positions is to be understood as covering at least a patient lying on their stomach (prone position), on their left or right side (lateral position), and on their back (supine position). There are two variants of the supine position: one in which the head is in a head coil and the other in which the head rests on a headrest pillow. It should be noted here that the head coil occupies a certain area above the eyes of the patient, which limits the possibilities for a video system.

The object of the disclosure is therefore to provide, for a magnetic resonance facility, a video playback facility that can be used as flexibly as possible, is of compact design, and is as magnetic-resonance-compatible as possible.

To achieve this object, a modular video playback facility for a patient on a patient table in a magnetic resonance facility is provided, comprising
at least one display module, comprising a respective display unit for the left and right eye of the head of the patient, wherein each display unit comprises
a display facility,
a support arrangement for holding the display facility at a distance from the head, e.g. laterally, when the display unit is in a use position, and
an optical arrangement mounted on the support arrangement, e.g. at least partially in front of the respective eye, for projecting onto the eye an image output by the display facility in the use position,
at least one holder which can be detachably connected to the display module and which has fastening means for detachably fastening the holder to at least one component arranged and/or arrangeable on the patient table,
a data transmission arrangement connectable to the display module, and
a power supply arrangement connectable to the display module.

In alternative embodiments, the display module can be in two parts, which means that the display units can be positioned separately, for example. However, it can e.g. be provided that both display units are incorporated into a single display module. For this purpose, it can be particularly advantageous for the support arrangements of both display units to be connectable or connected to a support facility of the display module by a connecting means. This improves handling and takes advantage of the fact that eyes are usually in a relatively fixed, pre-known spatial relationship to each other.

In order to facilitate a compact and low-profile design, the display facilities may e.g. be arranged adjacent to the head of the patient in the use position, while still allowing the patient to view them via the optical arrangements. There is usually some free space next to the head of the patient that can be utilized. This provides advantages over taller video systems, especially with the patient in the supine or prone position. Other embodiments are also conceivable in which the display facilities are located in front of the head of the patient. In principle, an arrangement behind the head of the patient is also conceivable, although a more complex optical arrangement may then be required.

In each case, the display facilities are at a distance from the head of the patient, e.g. so that there is no interference, or as little as possible, with an imaging process using the magnetic resonance facility. In other words, the use of the optical arrangements makes it possible for the display facilities to be located further away from the head of the patient to eliminate image artifacts as far as possible, for example if magnetic materials or the like are still present. For example, it can be provided that the distance between the display facility and the head in the use position and, for instance, also in the non-use position, is at least 5 cm, e.g. at least 10 cm.

The modular design makes it straightforward and easy to reconfigure the video playback facility, for example when the patient positioning is changed, by replacing the holder as appropriate. An advantageous development of the present disclosure thus provides that the video playback facility has a plurality of interchangeable holders for components assigned to different lying positions of the patient and/or fastening positions on at least one of the at least one component. For example, the holders for different patient positions can be designed differently, for example for attachment to a different component and/or in terms of their length, design, and shape. These specifically designed holders enable the proposed video playback facility to be used in all positionings of the patient.

In addition, handling is more convenient for users because the components can be made lightweight, are smaller overall, and the weight can be distributed between parts, for example between the power supply arrangement and the data transmission arrangement and the display module. In an embodiment, and as already mentioned, the vertical height can be limited, at least for the supine and prone positions, e.g. for all positions, so that usability is increased and less time and effort is required.

In an embodiment, it is provided that at least one of the at least one holder has a movement facility, e.g. a pivot facility, for moving the display module between the use position and at least one non-use position. In this way, the display module can be easily brought in front of the eyes of the patient and just as easily removed again. The non-use position is advantageously one in which the display module and/or the holder rests against the patient table and/or the component in a space-saving and unobtrusive manner and/or is even arranged at least partially recessed in a receptacle. In an embodiment, this allows the preparation time of the patient for an examination procedure to be kept short even when the video playback facility is used. The holder and/or the component can have locking means, e.g. provided by the movement facility, for locking the holder in the use position and/or the non-use position.

In an embodiment, the distance between the optical arrangement and the eye can be at least any suitable value, such as for instance one centimeter, at least 3 cm, ranging from 1 to 10 cm, and/or the optical arrangement and/or the support arrangement can be at least partially transparent. The advantage of this is that the patient can still at least partially perceive their surroundings and is not completely isolated from them. The free space between the optical arrangement and the eye ensures that part of the field of view is not blocked, e.g. by the respective support arrangement, so that the patient feels less confined. In addition, an at least partially transparent design of the optical arrangement, for example the use of an at least partially transparent mirror, at least in the field of view of the patient, can also enable the patient to still perceive their surroundings despite seeing images displayed on the display facility. Patient wishes in this regard can thus be met. Developments are also conceivable in which, if the patient actually wants to be completely isolated, at least one optionally deployable isolation element is provided as part of the display module and can be used accordingly. For example, the isolation element can be additionally mounted on the support arrangement and/or moved behind the optics or fixed there.

The display facilities can advantageously be or comprise an LCD display, an OLED display and/or a micro-LED display. In this way, available technologies are used that allow the display facilities to be made small, lightweight, and to require little electrical power for operation. This further contributes to a compact, easy-to-handle implementation and enables improved achievement of self-contained operation.

In some embodiments, it can be provided that the optical arrangement comprises at least one mirror, e.g. a parabolically shaped mirror, and/or at least one lens and/or at least one prism. For example, the optical arrangement can consist either of a lens or of a mirror or of a combination of these elements. In an embodiment, it is provided that the mirror is at least partially transparent, e.g. in the case of a laterally arranged display facility. If the respective display facility is arranged laterally, the mirror can, for example, be oriented at least approximately at an angle of 45°. In an embodiment, a partially transparent, parabolically shaped mirror is used, which simultaneously allows a view of the surroundings and projects the displayed image superimposed onto the retina of the patient. The mirror is located in front of the eye of the patient and deflects the optical path by its orientation angle, for example at least essentially 45°, onto the display facility. In embodiments with a mirror, the lens can be used, for example, to focus correctly on the eye.

In some embodiments, the data transmission arrangement and the power supply arrangement can be formed by at least one cable with a plug for the display module, possibly even integrated into a single cable. However, the video playback facility may be e.g. designed to be self-contained with respect to the power supply and data transmission facilities in the sense that no external power supply is required and the images to be played back are provided at least without a wired connection, e.g. not via a cable. In this way, magnetic resonance compatibility is increased and installation and use are simplified.

In an embodiment, the power supply arrangement can comprise a magnetic-resonance-compatible rechargeable battery and/or at least one photovoltaic module. This means that electrical power for operating the display facilities and, where applicable, the data transmission facility is locally available without the need for corresponding cabling. As already explained, the display module and, where necessary, the data transmission facility can be designed in such a way that only a small amount of power is required for their operation, so that simple, magnetic-resonance-compatible and space-saving elements of this kind are sufficient. The magnetic-resonance-compatible rechargeable battery may e.g. be designed largely without magnetic components. Such rechargeable batteries are commercially available, albeit at somewhat higher prices in some cases. For instance, the magnetic-resonance-compatible rechargeable battery may not have a protective plate made of stainless steel or similar, but rather a plastic protective housing and/or can be a lithium-ion battery.

Additionally or alternatively, it is also conceivable that the data transmission arrangement and/or the power supply arrangement comprise at least one cable and/or at least one plug, for example for connecting at least one assembly of at least one of these arrangements to the display module. However, It is also conceivable to receive from a remote source, via a cable with a plug, for example power and/or data relating to images for playback. For example, the plug can be designed to fit a coil connection socket in the patient table to receive electrical power and/or data there.

In embodiments, the data transmission arrangement can comprise an interface for the optical reception of images to be played back, e.g. a connection for a fiber-optic cable and/or a Li-Fi interface and/or an infrared link interface. Li-Fi (optical wireless transmission, Light Fidelity) is an optical wireless technology for data transmission. Unlike WLAN or other radio technologies, Li-Fi operates in the light spectrum - more precisely, using visible light or infrared radiation. The data transmission arrangement can therefore operate optically, specifically wirelessly via Li-Fi and/or infrared link. This variant is particularly advantageous when the video playback facility is used in a magnetic resonance facility. This is because in a cylindrical patient receptacle (often also referred to merely as a “bore”), the patient is always positioned on the patient table such that their head is at the head end or foot end of the patient table, and thus at one end of the receptacle. Consequently, there is always a direct, unobstructed line of sight from one end of the patient receptacle to the patient's head and thus to the data transmission arrangement (or a corresponding receive section of the data transmission arrangement). At least one of the light sources outside the patient receptacle, which radiate along the longitudinal axis of the receptacle, can therefore reach the head of the patient without interference.

In this context, an embodiment therefore provides that the magnetic resonance facility and/or a shielded enclosure for a main magnet unit housing the patient receptacle of the magnetic resonance facility comprises at least one optical transmission facility. For instance, in the case of a cylindrical patient receptacle, transmission facilities transmitting by means of Li-Fi and/or infrared, e.g. in a collimated and/or focused manner, can be provided on both sides in a direction along a longitudinal axis of the patient receptacle (z-direction). In the case of an end of the patient table that is generally located outside the patient receptacle (“foot end”), one of the transmission facilities can also be arranged there. In other words, this means that video data relating to images for playback can be transmitted optically, for example by means of a collimated light beam of comparatively low intensity, to a receive section of the data transmission arrangement in the region of the head of the patient. For this purpose, two optical transmission facilities are installed, one on each side outside the main magnet unit, for example on the walls of the shielded enclosure. Alternatively, at the foot end of the patient receptacle, the transmit facility can also be installed at the foot end of the patient table. The receive section is positioned accordingly in the region of the head of the patient.

Alternatively or additionally, it is also conceivable for the data transmission arrangement to comprise a radio interface and/or a galvanic interface for receiving images for playback, e.g. corresponding video data.

It should be noted at this point that the data transmission arrangement can also allow bidirectional data transmission, meaning e.g. that a receive section of the data transmission arrangement can also be designed as a transmit section. This may be particularly useful if the video playback facility and/or the component comprise further functional elements, such as sensors or the like, since measurement data can then also be output. However, the video playback facility can also be provided with control elements and/or other input means for the patient so that information can be transmitted from the patient to outside the patient receptacle. In addition, data other than images for playback can also be transmitted to the video playback facility, for example audio data for output by an audio output means forming part of the video playback facility and/or assigned thereto.

In an embodiment, the display units, e.g. at least their display facilities, and/or the power supply arrangement, and/or the data transmission arrangement, have a radio-frequency shield. In order to avoid interference with magnetic resonance imaging, the corresponding parts of the video playback facility that can potentially cause interference are advantageously provided with radio-frequency shields. The or each radio-frequency shield can consist at least partly of copper and/or aluminum and/or carbon and/or have, at least in part, a mesh structure. In an embodiment, it can be provided that the radio-frequency shield has at least partially a layer structure in which a metallic mesh layer, e.g. comprising copper, is arranged between two carbon-fiber layers. The mesh size of the mesh structure of the mesh layer can advantageously range from one to four millimeters. In this way, sufficient radio-frequency attenuation is achieved without causing disturbance in the main magnetic field (B0 field) and/or heating due to eddy currents in a solid radio-frequency shield.

A special case arises for the display facilities, since here the radio-frequency shield should have no effect, or at least as little effect as possible, on the recognizability of the images reproduced. In this context, an embodiment of the disclosure provides that the radio-frequency shield of the display facilities comprises at least one glass layer coated with ITO and/or silver and/or gold, e.g. a plurality of such glass layers, covering a display surface of the respective display facility. Accordingly, in the region of the display surface, i.e. where the images are played back, a special approach is taken to implement the shielding. Thus, embodiments include using ITO (indium tin oxide), e.g. to implement the radio-frequency shield on the front surface of the display facilities using ITO-coated glass to block radio-frequency interference. ITO is conductive and transparent to visible light, so that it has already been used in LCDs to form matrix contacts. Owing to these properties, it is used with particular advantage here as part of the radio-frequency shield. Alternatively, thin layers of silver or gold can be used, for example, but this is less preferred as it can impair the perceived image quality. In general, the coating, e.g. ITO, is galvanically connected at the edge of the glass layer to the rest of the radio-frequency shield. In order to achieve a higher radio-frequency density, preferred embodiments can provide that several, for example up to 10, coated glass layers per display facility are used as a stack, wherein each coating is in turn galvanically connected along the edge to the rest of the radio-frequency shield.

In exemplary embodiments, it can be provided that the power supply arrangement and the data arrangement are arranged at least partially as a common assembly, e.g. an assembly module, in a common housing. In this way, modularization can also be achieved to at least a certain extent with regard to these arrangements, for example in the form of an assembly providing at least a large part of the power supply arrangement and the data transmission arrangement, which can be connected to the display module via at least one, e.g. preferably exactly one, cable. Embodiments are also conceivable in which a plurality of such assemblies and/or additional individual assemblies for the data transmission arrangement and the power supply arrangement are provided and/or are at least partially integrated into the or a component.

In principle, it may be advantageous and is envisaged within the scope of the present disclosure to integrate at least part of the power supply arrangement and/or the data transmission arrangement into at least one of the at least one component. In this case, the component can be regarded as forming part of the video playback facility, or, conversely, the disclosure can also be regarded or formulated as relating to the corresponding component.

In an embodiment, it is provided that the component is a headrest pillow, e.g. forming part of the video playback facility, in which the power supply arrangement and the data transmission arrangement are at least partially integrated. In this case, a headrest pillow is provided in which parts of the video playback facility are already integrated and to which the display module is fastened or can be fastened by means of the holder. For instance, a plurality of holders can be provided for corresponding positionings of the patient. Specifically, it can be provided that the holder can be fastened to a side of the headrest pillow oriented laterally to the longitudinal axis of the patient table, e.g. in a lower, plate-like portion of the headrest pillow, and/or that the video playback facility comprises a first holder for a side-lying position of the patient for the lateral arrangement of the display module in the use position and at least a second holder for a supine position of the patient for arrangement of the display module above a support surface of the headrest pillow in the use position. In one embodiment, headrest-side fastening means can be used for both the first and at least one of the at least one second holder. For example, the same fastening points can be used. This simplifies orientation, handling, and correct attachment. In the embodiment in which a movement facility, e.g. a pivot facility, is provided, the use position can e.g. be a horizontal position of a support arm of the holder used, and the non-use position can be a horizontal alignment of the support arm.

In this context, an embodiment provides that the headrest pillow and/or the display module comprises a magnetic field sensor, e.g. a three-dimensional Hall sensor. In an embodiment, the display module can carry the magnetic field sensor, since it is arranged in a clearly defined position relative to the head, so that the position of the head can be easily inferred from the position data of the magnetic field sensor. It is also conceivable to provide a magnetic field sensor in an assembly of the power supply arrangement and/or the data transmission arrangement. In addition or alternatively, integration with the video playback facility can be achieved in such a way that the Hall sensor is supplied with power via the power supply arrangement and/or communicates via the data transmission arrangement.

Headrest pillows for a magnetic resonance facility in which a magnetic field sensor, e.g. a three-dimensional Hall sensor, is integrated are described, for example, in the later-published German patent application filed under 10 2024 205 084.6. These are used to provide information about the position of the head of the patient, as this is relevant for estimating the specific absorption rate (SAR), for which special limits may exist for the head. The magnetic field sensor measures e.g. the stray field of the magnetic resonance facility, which is characteristic of the position outside the patient receptacle. It has previously been proposed to integrate such magnetic field sensors into local coils to determine their position, see for example DE 10 2016 203 255 A1 and US 2017/0248665 A1. In each case, the aim is to determine a position in the coordinate system of the patient table by measuring the stray field. Once the type and extent of movement of the patient table are known, the respective position within the patient receptacle, for example relative to the imaging volume, can also be determined.

Since, by adding a magnetic field sensor, the headrest pillow ultimately becomes an electronic item, the addition of a video system to the headrest pillow, as described for the video playback facility according to the disclosure, is a logical development, just like the addition of the magnetic field sensor to the display module. In view of integrative aspects of product development, a combination of a video system, a headrest pillow, and a magnetic field sensor for determining the position of the head (with regard to SAR) is particularly advantageous. In this respect, the present disclosure can also be understood as relating to the headrest pillow.

Exemplary embodiments are also conceivable in which different headrest pillows are used for a side-lying position and/or a supine position. In this case, it can also be provided to integrate at least part of the power supply arrangement and/or the data transmission arrangement into all of the headrest pillows, which means that these components are provided multiple times.

However, within the scope of the present disclosure, fastening or integration is also conceivable with regard to other components that are or can be placed on the patient table, wherein e.g. at least the same display module can be used for all components. For example, it can be provided that one of the at least one holder, e.g. for the prone position of the patient, is provided for fastening to a head support element and/or a local coil facility serving as a component, and/or at least part of the power supply arrangement and/or the data transmission arrangement is installed in a local coil facility. For example, head support elements that are open in the middle are known, on which the patient can place their head when in the prone position. The or a holder can then also be fastened here, e.g. for the prone position, and/or at least parts of the data transmission arrangement and/or the power supply arrangement can be integrated there. Also advantageous is also integration with or fastening to a local coil facility, for example a breast coil, which in some cases may already include a head support element as just discussed. In the case of local coil facilities, an advantageous development can provide that the power supply and/or the data supply with images to be played back is provided via a coil plug of the local coil facility. In this case, therefore, not only the local coil facility but also the video playback facility is connected via the coil plug and the corresponding coil socket. For this purpose, it is of course necessary that the connections to the coil sockets are designed accordingly. In addition to a breast coil, which is particularly suitable for the prone position, it is also conceivable to provide fastening and/or at least partial integration with a head coil, wherein the video playback facility can then also be used in other positions of the patient, since the head coil often replaces the headrest pillow.

The video playback facility can also comprise, or be associated with, an audio output means. Embodiments have also already been proposed in which audio output means are integrated into a headrest pillow; of course, conventional headphones can also be used. For output, piezoelectric elements are preferably used to operate with minimal interference. The combination of video and audio enables, for example, films, series, music videos and the like to be played back.

The present disclosure also relates to a magnetic resonance facility comprising at least one video playback facility of the type according to the disclosure. All the embodiments relating to the video playback facility according to the disclosure may be applied analogously to the magnetic resonance facility according to the disclosure, so that the advantages already mentioned can also be obtained with the latter. For instance, in the case of wireless optical data transmission, the magnetic resonance facility can comprise the aforementioned transmission facilities. A control device can also be provided to select or specify data to be output by the video playback facility and, where applicable, simultaneously via the audio output facility.

### BRIEF DESCRIPTION OF THE DRAWINGS

Further advantages and details of the present disclosure will emerge from the exemplary embodiments described below and from the accompanying drawings in which:

FIG. 1 illustrates a schematic diagram of a magnetic resonance facility according to the disclosure;

FIG. 2 illustrates a first exemplary embodiment of the video playback facility according to the disclosure using a first holder;

FIG. 3 illustrates a schematic diagram of an example display module of the video playback facility;

FIG. 4 illustrates the first exemplary embodiment of the video playback facility according to the disclosure using a second holder; and

FIG. 5 illustrates a second exemplary embodiment of the video playback facility according to the disclosure.

### DETAILED DESCRIPTION OF THE DISCLOSURE

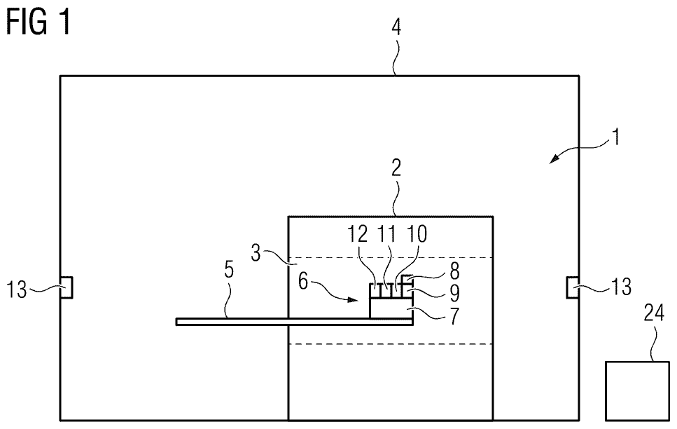

FIG. 1 illustrates a schematic diagram of a magnetic resonance facility according to the disclosure. More specifically, FIG. 1 shows a schematic diagram of a magnetic resonance facility 1 according to the disclosure, in which the main magnet unit 2, which contains the superconducting main magnet and has a cylindrical patient receptacle 3, is shown in greater detail. The main magnet unit 2 is housed in a shielded enclosure 4.

For examination, a patient can be moved into the cylindrical patient receptacle 3 by means of a patient table 5. For patient entertainment, a video playback facility 6 is provided which is fastened to or integrated with a component 7 which is or is to be arranged on the patient table 5. The component 7 can be a headrest pillow, a local coil facility or the like. An audio output device 8 is also provided as part of the video playback facility 6 or assigned thereto. As shown only schematically here, the video playback facility 6 comprises a display module 9, a holder 10, a power supply arrangement 11, and a data transmission arrangement 12. A plurality of holders 10 can be provided for different components 7 and/or different positionings of a patient.

Since in this example the data transmission arrangement 12 is designed for the use of wireless optical communication, two optical transmission facilities 13, which can emit collimated, low-energy light along the longitudinal axis of the patient receptacle 3 for Li-Fi communication and/or to establish an infrared link, are provided in the shielded enclosure 4. One of the transmission facilities 13 transmits from behind into the receptacle 3, the other from the front, so that, regardless of how the patient is positioned (head-first or feet-first), one of the transmission facilities 13 reaches the head area of the patient and thus the data transmission arrangement 12, specifically its receive section as an interface.

As mentioned above and will be explained in more detail below, the video playback facility 6 can comprise a plurality of holders 10 which are assigned to different positionings of the patient and, where applicable, also to different components.

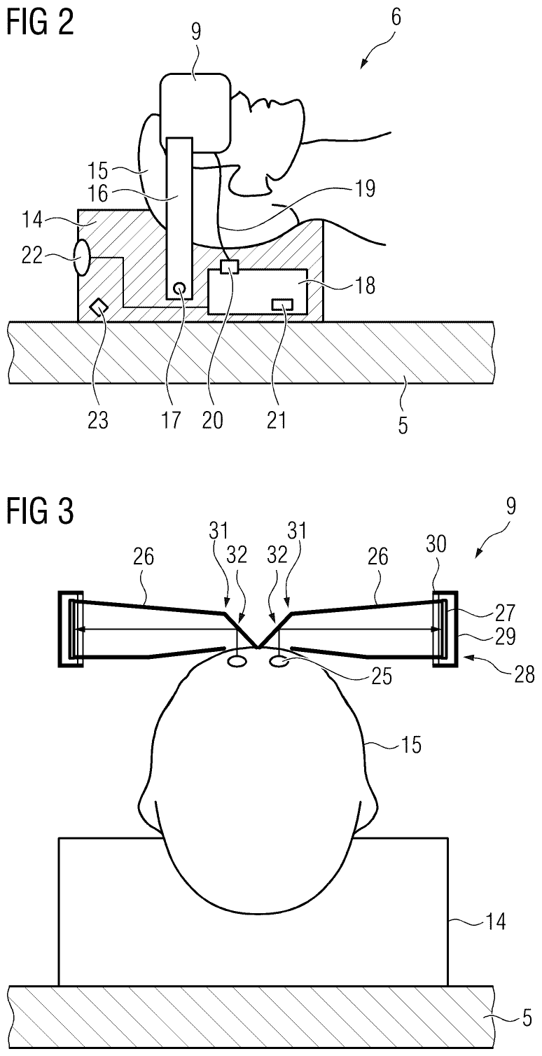

FIG. 2 illustrates a first exemplary embodiment of the video playback facility according to the disclosure using a first holder. More specifically, FIG. 2 shows a first specific example of a video playback facility 6. This is integrated with, or fastened to, a headrest pillow 14 as the component 7. The head 15 of the patient is shown in a supine position on the headrest pillow 14. The holder 10 comprises two holder arms 16 mounted on opposite sides of the headrest pillow 14 by means of appropriate fastening means (not shown) which support the display module 9 that extends above the head 15 of the patient in front of his eyes between the holder arms 16. By means of a movement facility 17, in this case a pivot facility, the support arms 16 can be moved with or without the display module 9 from the use position shown, in which the support arms 16 extend vertically, into a horizontal, non-obtrusive non-use position. Locking means, not shown in more detail, allow locking in both the use and non-use positions.

Parts of the power supply arrangement 11 and the data transmission arrangement 12 are in this case implemented in an assembly 18 installed in the headrest pillow 14. The assembly 18 is connected to the display module 9 via a cable 19 and a plug 20. As part of the power supply facility 11, the assembly 18 here comprises a magnetic-resonance-compatible rechargeable battery 21, for example a lithium-ion battery. Additionally or alternatively, photovoltaic modules to be used outside the assembly 18 can also be used as part of the power supply arrangement 11. The data transmission arrangement 12 here uses optical wireless transmission technology and thus comprises a receive section 22 oriented toward at least one of the transmission facilities 13, which receive section can comprise, for example, a lens and/or a photodiode and is connected to the assembly 18. The receive section 22 forms an interface for wireless optical data transmission.

Once the head 15 of the patient rests on the headrest pillow 14, the holder 10 is pivoted into the vertical use position and locked in place. If the display module has not yet been fastened to the holder 10 at this point, it can now be fastened to the holder 10 and the cable 19 can be connected to the assembly 18. The video playback facility 6 can now begin displaying images received via the data transmission arrangement 12.

As shown in FIG. 2, the headrest pillow here also comprises, integrated therein, a magnetic field sensor 23 implemented as a three-dimensional Hall sensor for example. This can also be supplied with electrical power via the power supply arrangement 11 and transmit its measurement data to a control facility 24 (see FIG. 1) of the magnetic resonance facility 1, which in turn can output images as video data and, where applicable, associated audio data for the audio output means 8. The receive section 22 is therefore also designed for transmission and the transmission facilities 13 are also designed for reception. In some embodiments, the magnetic field sensor 23 can also be part of the display module 9.

In an alternative variant, the data transmission and power supply to the headrest pillow 14 can also be provided galvanically via a coil cable and/or a dedicated cable system if the coil sockets do not provide audio and video data transmission.

FIG. 3 illustrates a schematic diagram of an example display module of the video playback facility. More specifically, FIG. 3 shows the display module 9 and its structure in more detail. This again shows the head 15 of the patient resting on the headrest pillow 14, with only the eyes 25 being indicated. Although the display module 9 is shown immediately adjacent to the head 15 for the sake of clarity, in practice it is located at a distance, e.g. at least one to ten centimeters, therefrom, for example. The display module 9 here has display units for the right and left eye, said display units being mirror-symmetrical and having support arrangements 26 (here provided as examples) that are connected to a common support facility by a connecting means (not shown) which may be detachable.

Each display unit primarily comprises a display facility 27 which can be designed, for example, as an LCD, OLED display, or micro-LED display, etc. The display facility 27 is surrounded by a radio-frequency shield 28, which in this case has two parts. The part 29 not covering the display surface of the display facility 27 consists of a plurality of carbon-fiber layers, between each of which a mesh structure or mesh layer made of copper is embedded, the mesh width of which may be one to four millimeters, for example. The part 29 is therefore a layer structure. At the side of the display surface, the radio-frequency shield 28 is impermeable to radio-frequency by means of ITO-coated glass 30 in a plurality of glass layers. Each ITO layer is galvanically connected along its edge to the part 29.

For the power supply arrangement 11 and the data transmission arrangement 12, parts causing interference to magnetic resonance imaging can also be provided with a radio-frequency shield 28 of this kind, in the form of the layer structure of the part 29.

Since the display facilities 27 are arranged laterally, e.g. at a 90° angle to the viewing direction, each display unit has an optical arrangement 31 which, in this case, comprises at least one parabolic, semi-transparent mirror 32 angled at least approximately at 45°. In addition, the optical arrangement 31 may also comprise a lens, not shown in detail here, for example for focusing. As can be seen, the optical path is deflected in a horizontal direction by means of the mirrors 32 and, where applicable, further optics, and ends at the display facilities 27 to the left and right of the head 15 of the patient. This results in a flat structure with respect to the vertical axis, so that the freedom of movement of the patient table 5 in the horizontal direction is not restricted. Also, the two display facilities 27 are located relatively far from the head 15 of the patient, for example at least 5 cm away, so that image artifacts can be avoided. Due to the distance (not shown in detail here) between the display module 9 and the eyes 25 and the partially transparent design of the mirrors 32, the patient is not completely isolated from their surroundings.

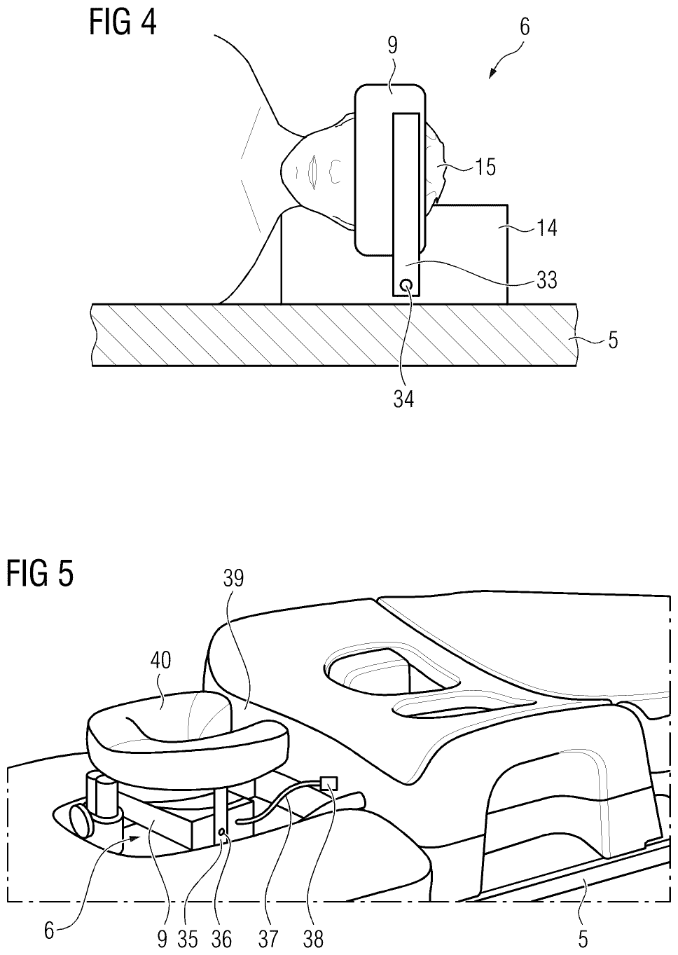

FIG. 4 illustrates the first exemplary embodiment of the video playback facility according to the disclosure using a second holder. More specifically, FIG. 4 illustrates how, using a different holder 10 with a holder arm 33 and a movement facility 34, the video playback facility 6 can also be used for a side-lying (lateral) position of the patient. FIG. 4 shows the left lateral position; for the right lateral position, a corresponding arrangement on the other side of the headrest pillow 14 can be used. Again, the use position is shown; for the non-use position, the support arm 33 can be pivoted into the vertical.

FIG. 5 illustrates a second exemplary embodiment of the video playback facility according to the disclosure. More specifically, FIG. 5 lastly shows a second exemplary embodiment of a video playback facility 6 according to the disclosure, having a display module 9 which is designed as shown in FIG. 3, and a holder 10 formed by a holding arm 35 and fastening means 36 for the display module 9, wherein the power supply arrangement 11 and the data transmission arrangement 12 are formed in this case by a cable 37 with a plug 38 that are connected to a breast coil 39 as the component 7. In this case, the video data and electrical power are supplied via the breast coil 39 inserted into a coil socket. However, optical wireless transmission is also essentially possible in this embodiment. Advantageously, further components of the power supply arrangement 11 and the data transmission arrangement 12 can be integrated within the breast coil 39.

The breast coil 39 here also comprises a head support element 40, to the carrier of which the holding arm 35 is fastened by appropriate fastening means. The embodiment shown in FIG. 5 is suitable for a patient in the prone position.

Independent of the grammatical term usage, individuals with male, female or other gender identities are included within the term.

Additionally, the various components described herein may be referred to as “facilities,” “modules,” or “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such facilities, modules, units, etc., as applicable and relevant, may alternatively be referred to herein as “circuitry,” “controllers,” “processors,” or “processing circuitry,” or alternatively as noted herein.

What is claimed is:

1. A modular video playback system for a patient on a patient table in a magnetic resonance device, the modular video playback system comprising
a display module comprising first and second display units for a left and right eye, respectively, of the patient, the first and second display units comprising:
a display;
a support arrangement configured to hold the display a distance from a head of the patient to facilitate the patient lying laterally on the patient table when the display module is in a use position; and
an optical arrangement fastened to the support arrangement at least partially in front of a respective left or right eye of the patient, the optical arrangement being configured to project an image output by the display onto the respective left or right eye of the patient;
a holder configured to be detachably connected to the display module and comprising a fastener configured to detachably fasten the holder to a component arranged on the patient table;
a data transmission arrangement configured to be connected to the display module; and
a power supply arrangement configured to be connected to the display module.

2. The video playback system as claimed in claim 1, further comprising:
a plurality of interchangeable holders for components assigned to different lying positions of the patient and/or fastening positions on the component.

3. The video playback system as claimed in claim 2, wherein the support arrangement of each of the first and second display units is configured to be connected to a support facility of the display module via a connecting means.

4. The video playback system as claimed in claim 1, wherein the holder of each of the first and second display units comprises a pivot facility configured to facilitate the display module being moved between the use position and a non-use position.

5. The video playback system as claimed in claim 1, wherein the power supply arrangement of each of the first and second display units comprises one or more of a magnetic-resonance-compatible rechargeable battery, a photovoltaic module, a cable, or a plug.

6. The video playback system as claimed in claim 1, wherein the data transmission arrangement of each of the first and second display units comprises an interface configured to play images optically received via one or more of a fiber-optic cable, a Li-Fi interface, or an infrared link interface.

7. The video playback system as claimed in claim 1, wherein one or more of the display, the power supply arrangement, or the data transmission arrangement of each of the left and right display units comprises a radio-frequency (RF) shield.

8. The video playback system as claimed in claim 7, wherein the RF shield at least partially comprises a layer structure in which a metallic mesh layer is arranged between two carbon-fiber layers.

9. The video playback system as claimed in claim 8, wherein the metallic mesh layer comprises copper.

10. The video playback system as claimed in claim 7, wherein the RF shield comprises at least one glass layer coated with one or more of indium tin oxide (ITO), silver, or gold.

11. The video playback system as claimed in claim 10, wherein the at least one glass layer is from among a plurality of glass layers covering a display surface of the display of each one of the first and second display units.

12. The video playback system as claimed in claim 1, wherein the power supply arrangement and the data transmission arrangement of each one of the first and second display units are at least partially arranged as a common assembly in a common housing.

13. The video playback system as claimed in claim 1, wherein the component comprises a headrest pillow in which the power supply arrangement and the data transmission arrangement, of each one of the first and second display units, are at least partially integrated.

14. The video playback system as claimed in claim 13, wherein the holder of each of the first and second display units is configured to be fastened to a side of the headrest pillow oriented laterally to a longitudinal axis of the patient table.

15. The video playback system as claimed in claim 13, wherein the holder of each of the first and second display units comprises:
a first holder configured to facilitate the patient lying laterally on the patient table by providing a lateral arrangement of the display module when the display module is in the use position; and
a second holder configured to facilitate the patient lying in a supine position by arranging the display module above a resting surface of the headrest pillow when the display module is in the use position.

16. The video playback system as claimed in claim 13, wherein the headrest pillow and/or the display module comprises a magnetic field sensor.

17. The video playback system as claimed in claim 16, wherein the magnetic field sensor comprises a three-dimensional Hall sensor.

18. The video playback system as claimed in claim 1, wherein the component arranged on the patient table is from among a plurality of components comprising a head support element and a local coil facility, and
wherein at least part of the power supply arrangement and/or the data transmission arrangement of each one of the left and right display units is integrated in the local coil facility.

19. The video playback system as claimed in claim 1, further comprising:
an audio output means for providing audio.

20. A magnetic resonance device, comprising:
a patient table configured to receive a patient to undergo a magnetic resonance examination; and
a modular video playback system, comprising
a display module comprising first and second display units for a left and right eye, respectively, of the patient, the first and second display units comprising:
a display;
a support arrangement configured to hold the display a distance from a head of the patient to facilitate the patient lying laterally on the patient table when the display module is in a use position; and
an optical arrangement fastened to the support arrangement at least partially in front of a respective left or right eye of the patient, the optical arrangement being configured to project an image output by the display onto the respective left or right eye of the patient;
a holder configured to be detachably connected to the display module and comprising a fastener configured to detachably fasten the holder to a component arranged on the patient table;
a data transmission arrangement configured to be connected to the display module; and
a power supply arrangement configured to be connected to the display module.
